(19) United States
(12) Patent Application Publication
(10) Pub. No.: US 2003/0228376 A1
(43) Pub. Date: Dec. 11, 2003

(54) Novel topical microbicidal compositions

(75) Inventors: Shirish Bhagwanlal Mody, Mumbai (IN); Madhukant Mansukhlal Doshi, Mumbai (IN); Mild Dattatraya Joshi, Mumbai (IN)

Correspondence Address: LACKENBACH SIEGEL, ONE CHASE ROAD, SCARSDALE, NY, 10583, US

(73) Assignee: J.B. Chemicals & Pharmaceuticals Limited, Mumbai (IN)

(21) Appl. No.: 10/163,940

(22) Filed: Jun. 6, 2002

(51) Int. Cl. A61K 33/36; A61K 31/4164

(52) U.S. Cl. 424/672; 514/396

(57) ABSTRACT

A pharmaceutical composition for topical application and manufacturing process thereof for treatment of microbial and mycotic infections caused by aerobic and anaerobic microorganisms is provided comprising metronidazole and Povidone-Iodine, in effective amounts. Such a composition can be administered topically to patients in need thereof in various pharmaceutical dosage forms.

### FIELD OF INVENTION

The present invention relates to pharmaceutical formulations for topical application and manufacturing processes therefor, which are suitable for the treatment of various topical infections.

### BACKGROUND OF THE INVENTION

Anaerobic bacteria are frequently found in infections of the skin, soft tissue, bones and in bacteremia. Injury to skin, bone or soft tissue by trauma, ischemia or surgery creates a suitable environment for anaerobic infections. Since the sites that are colonized by anaerobic bacteria contain many species of bacteria, disruption of anatomic barriers allows penetration of many organisms, resulting in mixed infections involving multiple species of anaerobes combined with facultative or microaerophilic organisms.

Two-thirds of clinically significant anaerobic infections involve following five anaerobes: Bacteroides fragilis group, Bacteroides melaninogenicus groups, Fusobacterium nucleatum, Clostridium perfringens, and anaerobic cocci.

Certain types of infections as stated below in Table 1 commonly involve anaerobic bacteria including lower extremity infections in diabetics or in patients with severe peripheral vascular disease.

TABLE 1

| Skin and soft | Incidence of anaerobic |
|---|---|
| tissue infections | involvement (%) |
| Diabetic foot ulcers | 95 |
| Infected diabetic gangrene | 85 |
| Non-clostridial crepitant cellulitis | 75 |
| Decubitus ulcer with bacteremia | 63 |
| Cutaneous abscesses | 62 |
| Soft tissue abscesses | 60 |
| Topical infection of head and neck | 48 |
| Topical infection of trunk | 36 |
| Topical infection of hand | 31 |
| Topical infection of buttock | 33 |

Therefore, there are many conditions such as diabetic ulcers, decubitus ulcers, cellulitis, pyoderma etc. that have aerobic and anaerobic microflora. Thus, it is rational to use an agent having action on both types of organisms.

U.S. Pat. No. 4,803,066 describes antibacterial and antifungal composition for topical application the composition comprise azole derivative with silver compound. Metronidazole 1% solution is reported to be effective in treating various ulcers which included pressure sores in elderly and chronically ill patients, diabetic ulcers, venous ulcers. The solution was also used as irrigation or packs in the management of ischiorectal abscess, large abscesses in other areas, undermining subcutaneous cavitation complicating simple sacral pressure sores. Metronidazole topical therapy is also recommended for anaerobic decubitus ulcers (Grade III & IV), marginal cellulitis and sacral ulcers.

U.S. Pat. No. 5,407,670 describes topical ointment for the treatment of epidermal trauma such as burns, rashes, lesions, wounds and decubital ulcers, which contains povidone-iodine along with polymyxin, bacitracin, neomycin, and sugar. U.S. Pat. No. 5,137,718 describes infection fighting composition for topical application containing povidone-iodine complex for viricidal or microbial agent.

Patients admitted in ICU, trauma ward, emergency wards, burn wards, unconscious patients, patients with neurological/spinal disorders and patients undergoing urinary tract surgery are often catheterized. Bladder irrigation with Povidone-Iodine is effective in prevention of urinary tract infection after single or intermittent catheterization.[Van Den Broek P J, Lancet, March, 1(8428), 5635, 1985].

Metronidazole is a bactericidal. It has activity against the facultative anaerobes Gardnerella vaginalis and Helicobacter pyroli and is effective against some spirochetes. Moreover, several protozoa and anaerobic bacteria including Bacteroides and Clostridium Spp. are sensitive to Metronidazole. Efficacy of metronidazole against obligate anaerobic bacteria in vitro including the gram-negative organisms Bacteroides fragilis, Fusobacterium Spp., Peptococcus Spp., Peptostreptococcus Spp., and Villanelle Spp. is well established.

The mechanism of action of Metronidazole is thought to involve interference with DNA by a metabolite in which a nitro group of metronidazole has been reduced by bacterial nitroreductases to an unstable intermediate, which interacts with DNA, effectively preventing further replication.

A Variety of nitroimidazoles are widely used as anti bacterial, antitrichomonal, anti-parasitic agents. Representative active agents are tinidazole, nimorazole, panidazole, flunidazole, ronidazole but metronidazole is the only one which is widely accepted in therapy.

Metronidazole is considered the ‘gold standard’ against which other antimicrobials with perceived anti-anaerobic activity are compared. This is due primarily to its killing of Baceroides spp., and the very low rate of resistance acquired by these bacteria.(Olsson-Liljequist B, Nord C E, Scand. J. Infect. Dis., 1981: Suppl. 26: 24-5, Aldridge K E, Gelfand M, Relier L B, et al; Aldridge K E, Gelfand M, Relier L B, et al; Diagn. Microbiol. Infect. Dis.; 1994; 18:235-41; Selkon J B, Scand. J. Infect. Dis., 1981: Suppl. 26: 19-23; Sigeti J S, Guiney Jr D G, Davis C E, J. Infect. Dis., 1983; 148:1083-9; Scher K. S., Surg. Gyn. Obstet., 1988: 167:175-9)

The cure rates in patients with intra abdominal infections such as Gangrenous or perforated appendicitis improvement occurred in 100% of the patients. (Willis A. T., Ferguson I. R., et al., B. Med. J., 1976: 1: 318-21; Foster M. C., Kaplia L et al., Rev. Infect. Dis., 1986: 8 Suppl.5: 5634-8.)

Metronidazole is much more in therapeutic use compared to other nitormidazoles. Metronidazole was marketed for therapeutic use in February 1960.

Metronidazole is the only nitroimidazole available for topical treatment. Metronidazole 0.8% gel is used for treatment of malodeorous fungating tumors, Decebitus ulcers and varicose ulcers. Metronidazole 0.75% cream is employed for treatment of rosacea.

Iodine has long been accepted as a uniquely effective antiseptic and used widely both for the prevention and treatment of infection. It has a broad antimicrobial spectrum: bacteria, viruses, bacterial endospores, fungi, and protozoas are destroyed, however, been limited by a number of undesirable factors. The disadvantages of iodine are an unpleasant odor and staining properties, unstability and irritation potential of solutions to animal tissue. Iodine solutions may prove toxic to open wounds.

An iodophor which is a complex of iodine in ionic or molecular form or both with a carrier that serves to increase the solubility of iodine in water and also provides a reservoir of iodine for a controlled and sustained release over time. There are two categories of iodophors, water-soluble and water insoluble. An example of a water-soluble iodophor is the polyvinylpyrrolidone-iodine complex widely used as a germicidal solution. An example of a water insoluble iodophor is polyvinyl alcohol sponge complexed with iodine, which can be used to wipe down and disinfect hard surfaces.

It was discovered that Povidone-Iodine [iodine complexed with the inert polymer, polyvinylpyrrolidone (povidone)] ceases to irritate, sensitize or stain and yet retains its unique microbicidal activity as iodine is continually delivered. Biochemical research has indicated that this high degree of microbiocidal activity is the result of the interruption of vital metabolic pathways. This is accomplished by the iodination of the amino acid sequence of the microorganisms' proteins. [Bloomfield S. F., “Chlorine & Iodine Formulations”, in Handbook of Disinfectants & Antiseptics, Ed. By Ascezi J. M., Marcel Dekker Inc., NY, 1996, pp 147-149]

Povidone-Iodine is effective against variety of strains such as Staphylococcus aureus, Proteus mirabilus, Proteus vulgaris, Escherichia coli, Enterobacter areogenes, Enterobacter Spp., Streptococcus faecalis, Streptococcus pyogenes, Streptococcus hemolyticus, Salmonella typhimurium, Salmonella typhosa, Salmonella type C1, Salmonella Spp., Serratia marcescens, Serratia Spp., Shigella sonni, Pseudomonas aeruginosa, Klebsiella pneumoniae, Diplococcus pneumoniae, Mycobacterium tuberculosis, Bacillus subtillis, Clostridium septicum, Clostridium tetani, Bacillus subtillis spores, Trichophyton rubrum, Candida albicans, richomonas vaginalis, Aspergillus flavus, Aspergillus niger.

Povidone-iodine is used for the treatment of burns and of different skin lesions (decubitus and leg ulcers, etc.). In special preparations it is available for the therapy of inflammations in the mouth and pharynx and for vaginitis. Povidone-Iodine is used in the treatment of skin disinfection in the prevention of nosocomial infections, especially, prior to invasive procedures such as the insertion of peripheral catheters, treatment of exit site infection [Tanaka S., Advances in Peritoneal Dialysis, 12, 214-7, 1996] and bacteraemia in haemodyletic patients [Fong I. W., Postgraduate Medicinal Journal, 69, Suppl 3S15-7, 1993]. It is also used as surgical scrub as an effective method for avoiding intra as well as post-surgical infection. [Tucci V. J., Stone A. M., Thompson C., Isenberg H. D., Wise L, Surg. Gynecol. Obstet., 145(3), 415-6,1977] Povidone-iodine cream effectively limits bacterial infection in patients with traumatic lacerations requiring sutures. [Gravett et al, Annals of Emergency Medicine, 16(2), 167-71, 1987].

Water soluble iodophors forms micellar aggregates which enables a reduction in the concentration of free available iodine in water as well as simultaneous reduction in the disadvantages of iodine i.e. its unpleasant odor, irritation and staining of tissue, and corrosion of metal surfaces. An important factor in creating an iodophor is that one wishes to keep the concentration of free iodine in the solution as low as possible; to be effective

### SUMMARY OF THE INVENTION

Thus, taking into consideration the limitations associated with the conventional topical composition with individual active agents stated above, the present inventor has discovered a composition comprising of an iodophor and a alkyl imidazole, which has a wide antimicrobial activity against aerobic as well as anaerobic bacteria. Preferably, the composition comprises metronidazole and povidone-iodine. Povidone-Iodine acts against aerobic organisms and metronidazole acts against anaerobic organisms.

The present invention provides formulations and methods for the treatment of individuals affected with various skin infections and injuries, such as pre-operative and post-operative antisepsis, diabetic ulcers, lapromatous ulcers, decubitus ulcers, cellulitis, and other skin infection showing the presence of both aerobic and anaerobic microorganisms, as well as wounds, such as contaminated lacerations, accidental wounds, traumatic wounds, abrasions, thermal wounds (Burns of 1st, 2nd, 3rd degree), and animal and human bites. The present invention also provides formulations and methods for the treatment of mycotic infections such as pyoderma, otitis externa, tinea pedis, tinea cruris, tinea corporis, tinea versicolor, and cutaneous candidiasis, topical treatment of monialliasis, trichomoniasis, and non-specific vaginitis. Moreover, the present invention provides formulations and methods for prophylactic treatment of patients, such as bladder irrigation during catheterisation and before catheter removal, and as a disinfectant in small surgical procedures, and in catheter (peritoneal/dialysis) exit site wounds.

None of the references mentioned earlier in the text teach the combination of metronidazole and povidone-iodine for treatment of microbial and mycotic infections caused by aerobic as well as anaerobic microorganisms. It is a object of this invention to provide a pharmaceutical formulation comprising combination of metronidazole and Povidone-Iodine in the form of topical pharmaceutical composition having the effect on aerobic and anaerobic bacteria. This combination has been found to be therapeutically advanced over either metronidazole or Povidone-Iodine individually with improved patient compliance.

The combination offers following advantages:

Easy application schedule i.e. single application takes care of both the types i.e. aerobic and anaerobic organisms.

Reduced number of applications.

Broad spectrum of anti microbial activity

Rapid control of infection.

This formulation when applied on the affected part, flows and fills out the wounded area after application and thereafter comes into contact with the damaged tissue with microbial infection. Metronidazole exerts its aerobicidal activity and Povidone-Iodine reacts with amino acids of microbial cell wall of anaerobic bacteria present thereby killing the microbes. Thus, the combination comprising Metronidazole and Povidone-Iodine is therapeutically better over either metronidazole or Povidone-Iodine individually. The combination has a topical microbicidal activity against bacteria including spores, fungi, yeast, protozoa and viruses, even in presence of blood, serum, pus and necrotic tissue.

### DETAILED DESCRIPTION OF THE INVENTION

The present invention is a pharmaceutical composition for topical application comprising of an iodophor and a alkyl imidazole, which has a wide antimicrobial activity against aerobic as well as anaerobic bacteria. Preferably, the composition comprises metronidazole and povidone-iodine. Povidone-Iodine acts against aerobic organisms and metronidazole acts against anaerobic organisms.

The term “pharmaceutical composition for topical application”, as used herein, means various compatible dosage forms which are suitable for administration to a human or veterinary application.

Suitably, the compositions is adapted for topical administration which include for instance, ointments, solutions, creams or lotions, powder, topical patches, aerosols and can be used in the form of scrub, irrigating solution and paint. In addition, compositions of the present invention may be used in impregnated dressings. Compositions of the present invention may also contain appropriate conventional additives such as preservatives, chelating agents, solvents to assist drug penetration and emollients, hydrocarbon waxes, oleaginous substances, fatty acids and fatty alcohol in ointments and creams. Ingredients present in the topical carrier of the present invention are suitable for administration to different infected sites.

Such a preparation is most preferably administered in the form of ointment and solution although the other dosage forms are also advantageously envisioned. Advantages to administering the composition as a ointment and solution include convenience, ease of application, increased safety.

Preferred pharmaceutical compositions for topical application according to the present invention comprises of metronidazole or a pharmaceutically acceptable salt or ester thereof from 0.01 to 10%, preferably from 0.05 to 5% and most preferably 1% by weight of the composition.

Metronidazole, i.e., 1-(beta-hydroxyethyl)-2-methyl-5-nitroimidazole, belongs to the class of alkyl imidazole derivatives and are useful as antimicrobial agents. The term “metronidazole,” as used in this specification and claims, includes not only 1-(2-hydroxyethyl)-2-methyl-5-nitroimidazole, but also those analogs and derivatives of metronidazole (salts, esters etc) that are soluble in the pharmaceutical compositions described herein and which exhibit therapeutic activity when applied as taught by the present invention.

A preferred pharmaceutical compositions for topical application according to the present invention comprises of Povidone-Iodine from 1 to 20%, preferably from 3 to 10% and most preferably 5% by weight of the composition.

Specific examples of iodophors useful in this invention include polyvinylpyrrolidone-iodine, polyvinyl alcohol-iodine, polyvinyl oxazolidone-iodine, polyvinyl imidazole-iodine, polyvinyl morpholone-iodine, polyvinyl caprolactam-iodine, soluble starch-iodine, betacyclodextrin-iodine, polyoxyethylenepolyoxypropylene condensate-iodine, and ethoxylated linear alcohol-iodine, with polyvinyl pyrrolidone-iodine being the most preferred. The iodophor as mentioned in the present invention is characterized by enhanced bactericidal, germicidal and other biocidal activity, and reduced vapor pressure and odor. Staining is virtually non-existent and wide dilution with water is possible.

The preferred pharmaceutical composition of the present invention is in the form of ointment comprising metronidazole and povidone-iodine impregnated in a suitable water soluble base. The means of formulating water soluble ointment bases are known to those skilled in the art. A water soluble base lowers surface tension of the composition aiding uniform distribution of the composition.

Water soluble bases are prepared from mixtures of high and low molecular weight polythylene glycols, which have general formula HOCH₂[CH₂OCH₂]ₙCH₂OH. Suitable derivatives include ethers and esters of the poly (substituted or unsubstituted alkylene) glycols, such as macrogol ethers and esters e.g. cetomacrogol; glycofurol; block copolymers including poly (substituted or unsubstituted alkylene) glycols such as block copolymers of polyethylene glycol and polypropylene glycol and cross-linked polyethylene glycols.

Various grades of poly (substituted or unsubstituted alkylene) glycols and derivatives thereof may be used in combination to achieve the desired physical properties of the formulation. Preferably the formulation comprises polyethylene glycol or a derivative thereof which are commercially available in a variety of chain lengths and with a variety of consistencies. Suitable polyethylene glycols include PEG 300 and PEG 400 (liquids); PEG 1000 (semi-solids); and PEG 4000 and PEG 6000 (hard solids).

These may be used singly or admixed in suitable proportions to achieve the desired consistency of formulation. A preferred combination comprises PEG 4000 and PEG 400, suitably in a ratio of from 0.5:1 to 1:5, preferably from 1:1 to 1:3; most preferably about 1:2.

Typically, the vehicle comprises at least 70%, preferably at least 80%, most preferably at least 90% by weight of a pharmaceutically acceptable poly (substituted or unsubstituted alkylene) glycol or a derivative thereof.

Where the pharmaceutical composition is in the form of solution the active ingredients are combined with following ingredients:

Surface active agent

Co-solvent

Buffering agent

The expression “Surface active agent” as used in this specification refers to anionic surfactant. Such a sufactant provides better surface contact of the composition with infected area.

Specific preferred anionic surfactants include, but are not limited to, lauryl sulfates, octyl sulfates, 2-ethylhexyl sulfates, decyl sulfates, tridecyl sulfates, cocoates, lauroyl sarcosinates, lauryl sulfosuccinates, diphenyl oxide disulfonates, lauryl sulfosuccinates, myristyl sulfates, oleates, stearates, tallates, ricinoleates, cetyl sulfates, and similar surfactants.

However, sodium lauryl sulphate is preferably used as a surface active agent in the solution composition of the present invention in an amount of 0.1% to about 5.0% by wt. and preferably, in an amount of about 0.5% by wt. based on the total wt. of the composition.

The expression “co-solvent” as used in this specification refers to used in combination to increase the solubility of the solutes. Examples of preferred class are ethanol, sorbitol, glycerin, propylene glycol and members of polyethylene glycol polymer series. However, Polyethylene glycol 400 is preferably used as a cosolvent in the solution composition of the present invention in an amount of 2.5% to about 10.0% by wt. and preferably, in an amount of about 5.0% by wt. based on the total wt. of the composition.

The expression buffering agent as used in this specification refers to combination of basic pH adjuster and acidic pH adjuster.

Examples of preferred classes of basic pH adjusters are ammonia; mono-, di- , and tri-alkyl amines; mono-, di-, and tri-alkanolamines; alkali metal and alkaline earth metal hydroxides; alkaline phosphates and mixtures thereof. However, the identity of the basic pH adjuster is not limited, and any basic pH adjuster known in the art can be used. However, Dibasic sodium phosphate is preferably used as basic pH adjuster in the solution composition of the present invention in an amount of 2.5% to about 5.0% by wt. and preferably, in an amount of about 3.83% by wt. based on the total wt. of the composition.

The preferred classes of acidic pH adjusters are the mineral acids and polycarboxylic acids. Examples of mineral acids are hydrochloric acid, nitric acid, phosphoric acid, and sulfuric acid. Nonlimiting examples of polycarboxylic acids are citric acid, glycolic acid, and lactic acid. The identity of the acidic pH adjuster is not limited and any acidic pH adjuster known in the art, alone or in combination can be used. However, Citric acid is preferably used as acidic pH adjuster in the solution composition of the present invention in an amount of 0.5% to about 2.0% by wt. and preferably, in an amount of about 1.63% by wt. based on the total wt. of the composition.

The most preferred composition has a pH of below 7, most preferably between 5 to 6.5.

The pharmaceutical composition of the invention in the form of ointment can be prepared as follows: Metronidazole is dissolved in a mixture of PEG 400 and water under stirring. Then Povidone-Iodine is added to above solution and dissolved under stirring. Then PEG 4000 is melted by heating to 60-65° C. and then added to the above viscous solution under stirring. The mixture is allowed to cool to room temperature to form uniform viscous ointment.

The pharmaceutical composition of the invention in the form solution can be prepared by the method stated below:

The buffer is prepared by dissolving dibasic sodium phosphate and citric acid in water. Povidone-Iodine is dissolved in buffer under stirring. Metronidazole is dissolved in PEG 400 under stirring and added to the above solution containing Povidone-Iodine with mixing. Sodium lauryl sulphate is dissolved in water and added to the bulk solution under stirring. The volume is adjusted with water to get the specified concentration.

To investigate the effectiveness of the present invention in various types of wounds, controlled clinical trials were carried out all over India.

This study is not disclosed to the public and the trials are done in confidence. The results of clinical study in India are given below.

40 patients having lacerated wound were included in the study to evaluate the efficacy and safety of Metronidazole and Povidone-Iodine ointment as described in present invention and its comparison with Povidone-Iodine ointment 5%. Patients were divided in to two groups of twenty each. Group one received treatment with Povidone-Iodine ointment 5% where as group two received treatment with Metronidazole and Povidone-Iodine ointment as described in present invention. General and wound parameters such as pain, tenderness, edema, discharge, stages of healing, final healing, type and strength of scar were recorded. Treatment was given twice a day in each group. In group one healing took place in 8 weeks where as in group 2 it took 5 weeks. The improvement in pain, tenderness, edema and discharge improved much faster in Metronidazole and Povidone-Iodine ointment group as described in present invention group compared to Povidone-Iodine 5% group. Similarly scar formation was much faster in Metronidazole and Povidone-Iodine ointment as described in present invention group than Povidone-Iodine 5% ointment.

50 patients suffering from Bacterial and mycotic skin infections were included in the trial. They were divided in to two groups 25 each. Group 1 received treatment with Povidone-Iodine ointment 5% and group 2 received Metronidazole and Povidone-Iodine ointment as described in present invention ointment. Both the ointments applied twice a day. The time for recovery, signs of inflammation and response of the lesions were monitors. All patients completed study without any side effect. The healing of lesions in Povidone-Iodine ointment 5% group occurred in 9 days while in Metronidazole and Povidone-Iodine ointment as described in present invention ointment group healing occurred in 6 days. Inflammatory parameters showed faster remission in Metronidazole and Povidone-Iodine ointment as described in present invention group than Povidone-Iodine ointment 5% group.

50 patients undergoing gastrointestinal surgery were included in the evaluation of Metronidazole and Povidone-Iodine solution as described in present invention 5% solution and its comparison with Povidone-Iodine 5% solution as pre operative and post-operative anti-sepsis. They were divided two groups of 25 each group 1 received treatment with Povidone-Iodine 5% solution as pre and post operative scrub and Povidone-Iodine 5% ointment post operatively applied twice a day on operation wound. Group 2 received Metronidazole and Povidone-Iodine solution as described in present invention 5% solution as pre and post-operative scrub and Metronidazole and Povidone-Iodine solution as described in present invention 5% ointment as application twice a day on surgical wound. There were no serious post operative wound infections in any of the group. However, healing of the wound was much faster in Metronidazole and Povidone-Iodine solution as described in present invention group than Povidone-Iodine 5% solution group.

30 patients undergoing gastrointestinal surgery were included in the evaluation of Metronidazole 1% gel and Metronidazole and Povidone Iodine ointment. 30 patients were divided in to group of 15 each Group I received Metronidazole 1% Gel in form of topical application over incision post-operatively and Group II received Metronidazole and Povidone Iodine ointment applied topically on incision post-operatively. The dressing in both the group done daily and ointment and gel were applied twice daily. There were no serious post-operative wound infection in any of the group however healing of the wound was faster in Metronidazole and Povidone Iodine ointment group compared to Metronidazole 1% gel group.

Above clinical studies confirm the efficacy of the present pharmaceutical composition of this invention:

From this trial it can be concluded that Metronidazole and Povidone-Iodine as described in present invention is better than Povidone-Iodine alone in the management of bacterial and mycotic skin infections. This can be attributed to the unique combination comprising Metronidazole, an anaerobicidal agent and Povidone-Iodine, an aerobicidal agent which offered significantly rapid reduction due to the synergistic effect. This can be attributed to the unique combination comprising metronidazole, an anaerobicidal agent, and povidone-iodine, an aerobicidal agent, that offers significant rapid reduction of infection due to their combined action, which increases the effectiveness of each other.

The invention will now be illustrated by the following Examples:

### EXAMPLE 1

| | |
|---|---|
| Metronidazole | 1.00% |
| Povidone-Iodine | 5.00% |
| Polyethylene glycol 4000 | 30.00% |
| Polyethylene glycol 400 | 59.75% |
| Purified Water | 4.25% |

The ointment preparations of the invention can be prepared by dissolving Metronidazole in a mixture of PEG 400 and water under stirring. Then adding Povidone-Iodine to above solution and dissolving under stirring. Then melting PEG 4000 by heating to 60-65° C. and adding to the above viscous solution under stirring. Allowing to cool to room temperature to form uniform viscous ointment.

| | |
|---|---|
| Metronidazole | 2.00% |
| Povidone-Iodine | 10.00% |
| Polyethylene glycol 4000 | 30.00% |
| Polyethylene glycol 400 | 59.75% |
| Purified Water | 4.25% |

The same procedure as used in Example 1 was repeated only change is the concentration of the metronidazole and Povidone-Iodine are different to that of Example 1.

| | |
|---|---|
| Metronidazole | 1.00% |
| Povidone-Iodine | 5.00% |
| Polyethylene glycol 400 | 5.00% |
| Sodium lauryl sulphate | 0.50% |
| Dibasic Sodium phosphate | 3.83% |
| Citric acid | 1.63% |
| Purified Water | 4.25% |

The solution preparation of this invention can be prepared by dissolving dibasic sodium phosphate and citric acid in water. In this solution dissolving Povidone-Iodine under stirring. Then dissolving metronidazole in PEG 400 under stirring and adding this solution to the above solution containing Povidone-Iodine. Mixing well. Then dissolving sodium lauryl sulphate in water and adding this to the bulk solution under stirring. Mixing well and adjusting the volume with water to get the specified concentration.

| | |
|---|---|
| Metronidazole | 2.00% |
| Povidone-Iodine | 10.00% |
| Polyethylene glycol 400 | 5.00% |
| Sodium lauryl sulphate | 0.50% |
| Dibasic Sodium phosphate | 3.83% |
| Citric acid | 1.63% |
| Purified Water | 4.25% |

The same procedure as used in example 3 was repeated only change is the concentration of the metronidazole and Povidone-Iodine are different to that of example 3.

In addition the combination of Metronidazole and Povidone-Iodine may be applied or formulated contemporaneously with other topical agents to provide synergistic or amplified activity for management of wounds.

It is to be understood that the example and embodiments described hereinabove are for the purpose of providing a description of the present invention by way of example and are not to be viewed as limiting the present invention in any way. Various modifications or changes that may be made to that described hereinabove by those of ordinary skill in the art are also contemplated by the present invention and are to be included within the spirit and purview of this application and the stated claims.

What is claimed is:

1. A pharmaceutical composition for topical application suitable for treatment of various types of wounds comprising therapeutically effective amounts of alkyl imidazole derivative and iodophor as active ingredients.

2. A pharmaceutical composition for topical application as stated in claim 1 may be applied in the form selected from ointments, creams or lotions, solution, powder, patches and aerosols; most preferably in the form ointment and solution.

3. A pharmaceutical composition for topical application comprising therapeutically effective amounts of alkyl imidazole derivative and iodophor as active ingredients as claimed under claim 1 is suitable for treatment of various types of microbial and mycotic infections caused by aerobic and anaerobic microorganisms.

4. A pharmaceutical composition for topical application in accordance with claim 1 used for the treatment of contaminated lacerations, accidental wounds, traumatic wounds, thermal wounds (burn), animal and human bites, diabetic ulcers, decubitus ulcers, cellulitis, pyoderma, pre-operative and post operative antisepsis, or catheter exit site wounds.

5. A pharmaceutical composition for topical application suitable for treatment of various types of wounds in accordance with claim 1 wherein the alkyl imidazole derivative is preferably 5-nitroimidazole derivatives such as tinidazole, nimorazole, satranidazole, ornidazole, metronidazole and benznidazole.

6. The most preferred nitroimidazole useful in this invention as stated under claim 4 is Metronidazole or a pharmaceutically acceptable salt or ester thereof.

7. A pharmaceutical composition for topical application suitable for treatment of various types of wounds in accordance with claim 1 wherein the iodophor is selected from the group consisting of polyvinylpyrrolidone-iodine, polyvinyl alcohol-iodine, polyvinyl oxazolidone-iodine, polyvinyl imidazole-iodine, polyvinyl morpholone-iodine, and polyvinyl caprolactam-iodine, nonylphenolethoxylate-iodine, soluble starch-iodine, betacyclodextrin-iodine, polyoxyethylenepolyoxypropylene condensate-iodine, ethoxylated linear alcohol-iodine, and mixtures thereof.

8. The most preferred iodophor useful in this invention as stated under claim 5 is polyvinyl pyrrolidone-iodine.

9. A pharmaceutical composition for topical application in accordance with claim 1 comprising
from 0.01 to 10% of metronidazole or a pharmaceutically acceptable salt or ester thereof by weight of the composition; and
from 2 to 20% Povidone-Iodine by weight of the composition.

10. A pharmaceutical composition for topical application in accordance with claim 1 comprising
from 0.05 to 5% metronidazole or a pharmaceutically acceptable salt or ester thereof by weight of the composition; and
from 5 to 15% Povidone-Iodine or a pharmaceutically acceptable salt thereof by weight of the composition.

11. The composition as claimed in claim 1 which comprises metronidazole, present in about 1% by weight of the composition and the Povidone-Iodine is present in about 5% (i.e. 0.5% as the available iodine) by weight of the composition.

12. The composition as claimed in claim 1, wherein the Metronidazole and Povidone-Iodine are incorporated into the pharmaceutical formulation(s) suitable for topical application.

13. A pharmaceutical composition for topical application in accordance with claim 2 in the form of ointment contains a combination comprising of PEG 4000 and PEG 400, suitably in a ratio of from 0.5:1 to 1:5, preferably from 1:1 to 1:3; more preferably about 1:2.

14. A pharmaceutical composition for topical application in accordance with claim 2 in the form of solution contains a surface-active agent, buffering agent and a cosolvent.

15. A pharmaceutical composition for topical application in the form of solution in accordance with claim 12 the said surface active agent is selected from lauryl sulfates, octyl sulfates, 2-ethylhexyl sulfates, decyl sulfates, tridecyl sulfates, cocoates, lauroyl sarcosinates, lauryl sulfosuccinates, diphenyl oxide disulfonates, lauryl sulfosuccinates, myristyl sulfates, oleates, stearates, tallates, ricinoleates, cetyl sulfates, and similar surfactants.

16. A pharmaceutical composition for topical application in the form of solution in accordance with claim 12, the preferred surface active agent is sodium lauryl sulphate present in an amount of 0.1% to about 5.0% by wt. and preferably, in an amount of about 5.0% by wt. based on the total wt. of the composition.

17. A pharmaceutical composition for topical application in the form of solution in accordance with claim 12 the said buffering agent is a combination of basic pH adjuster and acidic pH adjuster.

18. A pharmaceutical composition for topical application in the form of solution in accordance with claim 15, further comprising a basic pH adjuster in an amount of 2.5% to about 5.0% by wt. based on the total wt. of the composition, and an acidic pH adjuster in an amount of 0.5% to about 2.0% by wt.

19. A pharmaceutical composition for topical application in the form of solution in accordance with claim 12 the said co-solvent is selected from ethanol, sorbitol, glycerin, propylene glycol and different grades of polyethylene glycol polymers.

20. A pharmaceutical composition for topical application in the form of solution in accordance with claim 12 the said co-solvent is polyethylene glycol 400 present in an amount of 2.5% to about 10.0% by wt. and preferably, in an amount of about 5.0% by wt. based on the total wt. of the composition

21. A process for preparation of pharmaceutical composition for topical application in the form of ointment according to claim 2 as described in detail in the text.

22. A process for preparation of pharmaceutical composition for topical application in the form of solution according to claim 2 as described in detail in the text.
